SEQUENCE LISTING (22 sequences)

SEQ ID NO: 1 - protein, 472 aa, synthetic construct
MVVGILATLATLATLAASVPLEERQSCSSVWGQCGGQNWAGPFCCASGSTCVYSNDYYSQCLPGAASSSSSTRASSTTSRVSSATSTRSSSSTPPPASSTTPAPPVGSGTATYSGNPFAGVTPWANSFYASEVSTLAIPSLTGAMATAAAAVAKVPSFMWLDTLDKTPLMSSTLSDIRAANKAGGNYAGQFVVYDLPDRDCAAAASNGEYSIADGGVAKYKNYIDTIRGIVTTFSDVRILLVIEPDSLANLVTNLATPKCSNAQSAYLECINYAITQLNLPNVAMYLDAGHAGWLGWPANQDPAAQLFANVYKNASSPRAVRGLATNVANYNAWNITTPPSYTQGNAVYNEKLYIHALGPLLANHGWSNAFFITDQGRSGKQPTGQLEWGNWCNAVGTGFGIRPSANTGDSLLDSFVWIKPGGECDGTSNSSAPRFDYHCASADALQPAPQAGSWFQAYFVQLLTNANPSFL

SEQ ID NO: 2 - DNA, 1419 bp, synthetic construct
  source 1..1419: mol_type = other DNA
atggttgtaggcattcttgcgactctggccacgctggccacactggcagctagcgttccccttgaggaacgacagtcttgctcaagtgtttggggtcaatgtggtggccaaaactgggctggaccgttctgctgtgcttccggaagcacttgtgtgtattccaatgactactactctcagtgtcttcctggcgcggcaagctcaagttcttccactcgcgcctcgtcgacaacttctcgggtgtcgtcagcaacatcgactagatcaagctcttcaacgccccctcctgcctcttcaactacaccagcgccgccagtcggatcgggaacggcaacatactccggtaacccctttgctggagttacgccttgggccaattccttttatgcctctgaagtcagcactttggcaattccaagcttgacaggagctatggccactgctgcggccgctgtggcaaaggtgccgtctttcatgtggctagacactctggataagactcctttgatgagctcgacgttgtcggacatccgtgccgcaaacaaggccggcggcaactacgctggacaatttgtcgtctacgacctgccagaccgtgactgtgccgccgctgcttctaatggtgaatattccatcgctgatggtggcgtggccaaatacaagaactatatcgatactattcgtggaatcgtcaccaccttttccgacgttcgcatacttttggttatcgagcccgactcccttgcgaacctggttactaacctcgccactccgaagtgctcaaacgcccaatctgcatacctggagtgcattaattacgcaatcacgcagctaaatcttcccaatgttgcgatgtacttggatgctggccacgctggatggcttggatggccagcaaatcaagacccagctgcccagttgtttgcaaatgtttacaagaacgcgtcatctcctcgagccgtccgtggactggccactaacgtggctaactacaatgcgtggaacattactaccccaccttcatacactcaaggaaacgctgtttacaatgagaagctgtatattcacgccctcggaccccttcttgccaaccatggctggtcaaacgctttcttcatcactgaccaaggccgatctggcaagcagccaaccgggcagcttgagtggggtaactggtgcaatgctgttggcactggatttggtatccgtccttctgccaacactggcgactcactgttggattcattcgtctggatcaagccaggtggcgagtgtgatggtactagcaacagcagtgcaccgcggtttgattatcactgtgcttctgcagatgctttgcagccagcacctcaagccggttcttggttccaggcttatttcgtgcagcttcttacgaacgcgaacccgtcattcctgtag

SEQ ID NO: 3 - protein, 226 aa, synthetic construct
MASSLVLIAAALLALASSWQQAIAYDPSPLQDFCVADKNSPVRVNGFPCKDPMAVTPDDFFNAAMIIDKRRDTNNKVGSNVTNVNVESFPGLNTLGISLARIDYAPLGVNPPHIHPRATELLTVLEGTLYLGFVTSNPNRLFSKVVKKGDVFVFPKAMIHFQMNLDHDKPAAALSSLSSQNPGVITIASAVFGSKPPISDDVLAKAFQVEKKLIDWLQSQFWDTNY

SEQ ID NO: 4 - DNA, 681 bp, synthetic construct
  source 1..681: mol_type = other DNA
atggcctcctccttggtcctcatcgcagcggccctgcttgcgctggcctcgtcgtggcagcaggccatcgcctacgatcctagccccctccaggacttctgtgtcgcagacaagaactcgcctgttcgcgtcaacgggttcccttgcaaggacccgatggcggtcaccccggacgacttcttcaacgcggcgatgatcatcgacaagcggagggacacgaacaacaaggtggggtccaacgtcaccaacgtgaacgtggagagcttcccggggctgaacacgctgggcatctcgctggcgcgcatcgactacgcgccgctcggggtgaacccgccgcacatccacccacgcgctaccgagctgctcaccgtgctcgagggcaccctctacctcggcttcgtcacgtccaaccccaaccgcctcttctccaaggtggtcaagaagggcgacgtcttcgtcttccccaaggccatgatccacttccagatgaacctcgaccacgacaagcccgccgcggcgctctcgtcgctcagcagccagaaccctggcgtcatcaccatcgccagcgccgtcttcggctccaagccgcccatctcggacgacgtcttggccaaggcgttccaggtggagaagaaactcatcgactggctccagtctcagttctgggataccaactactag

SEQ ID NO: 5 - DNA, 46 bp, synthetic construct
  source 1..46: mol_type = other DNA
caggtcgactctagaggatccgaattcatgccagttgttcccagtg

SEQ ID NO: 6 - DNA, 37 bp, synthetic construct
  source 1..37: mol_type = other DNA
ctacaaccatatcgatgcttgggtagaataggtaagt

SEQ ID NO: 7 - DNA, 51 bp, synthetic construct
  source 1..51: mol_type = other DNA
cggggcccagctaatggtgatggtgatgatgcaggaatgacgggttcgcgt

SEQ ID NO: 8 - DNA, 45 bp, synthetic construct
  source 1..45: mol_type = other DNA
tcattcctgcatcatcaccatcaccattagctgggccccggatcc

SEQ ID NO: 9 - DNA, 25 bp, synthetic construct
  source 1..25: mol_type = other DNA
tcaccattagctgggccccggatcc

SEQ ID NO: 10 - DNA, 57 bp, synthetic construct
  source 1..57: mol_type = other DNA
gatgtgttgacctccgaattctctagaaagaaggattacctctaaacaagtgtacct

SEQ ID NO: 11 - DNA, 20 bp, synthetic construct
  source 1..20: mol_type = other DNA
atggcctcctccttggtcct

SEQ ID NO: 12 - DNA, 30 bp, synthetic construct
  source 1..30: mol_type = other DNA
ctagtagttggtatcccagaactgagactg

SEQ ID NO: 13 - DNA, 1286 bp, synthetic construct
  source 1..1286: mol_type = other DNA
gaattcatgccagttgttcccagtgatcttcgtttcgaagatggacactcccaatttgtgcaagttattcggcctacctggctgtggccgaggcgcgttatcatgaccgtcgctgttcaaagataaggcgagaagtttgcgggctgtcttgacgatatggcttcgttcagacagatatagttcccggagtcgcaggcgtctattcttctccgaaacaaactcggctgcactgtttccatcaccgggtctggcgttgaggatgtcagcgaaactcggcccggcaagtgacacccgaaaagtatcgactccggctgcccgtttcaagctagtggcttcctcatcagcgagtcggccaaggagacgtgaagcaggacgggtttgccattccaagaccgtgatccgaagcgcgttgatttcatcaatcccagccttttcgctcaaccaaagagcatcggctttgatttccttcaggtcatacgaggcttgtgcaatggtctccgcatggatcgctgctgttctcctatcaaactcggattttgtcttaggggatggcgtaggaaagacgctgccgcggttcagaagcacctcgatgctatcaggatgtgacaaaaacgactcgaaaacccgggattcatcggtgatgctttcgggatcgcaagcgtaaagaaagactctcttccaagacctagaagtatagcaaaatcagcagcagaccatcaatgtatagcgaatgcgcccatacaaaagctgaacgtccccggagaagcacttgtccagggacgggaaataggcttccggaacgggagccattggcagcacagctatatcattctaagtaaacaaatgtaatgagcaagcggacggagtgctgaaacctccgtatgcctgaagccgacgaaagcgcgttggattagaggtcgacagaagatgatattgaaggagcactttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtgaacgacccggtcataccttcttaagttcgcccttcctccctttatttcagattcaatctgacttacctattctacccaagcatcgat

SEQ ID NO: 14 - DNA, 733 bp, synthetic construct
  source 1..733: mol_type = other DNA
ctgggccccggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcatgtttcttccttgaactctcaagcctacaggacacacattcatcgtaggtataaacctcgaaatcattcctactaagatggtatacaatagtaaccatggttgcctagtgaatgctccgtaacacccaatacgccggccgaaacttttttacaactctcctatgagtcgtttacccagaatgcacaggtacacttgtttagaggtaatccttctttctaga

SEQ ID NO: 15 - DNA, 26 bp, synthetic construct
  source 1..26: mol_type = other DNA
atgattgaacaagatggattgcacgc

SEQ ID NO: 16 - DNA, 24 bp, synthetic construct
  source 1..24: mol_type = other DNA
tcagaagaactcgtcaagaaggcg

SEQ ID NO: 17 - DNA, 20 bp, synthetic construct
  source 1..20: mol_type = other DNA
gttctctgccatcgctctct

SEQ ID NO: 18 - DNA, 20 bp, synthetic construct
  source 1..20: mol_type = other DNA
agcgccacagcacttgggga

SEQ ID NO: 19 - DNA, 24 bp, synthetic construct
  source 1..24: mol_type = other DNA
gtatcatgatcggtatgggtcaga

SEQ ID NO: 20 - DNA, 23 bp, synthetic construct
  source 1..23: mol_type = other DNA
tagaaggtgtggtgccagatctt

SEQ ID NO: 21 - DNA, 25 bp, synthetic construct
  source 1..25: mol_type = other DNA
atggcctcctccttggtcctcatcg

SEQ ID NO: 22 - DNA, 35 bp, synthetic construct
  source 1..35: mol_type = other DNA
ctagtagttggtatcccagaactgagactggagcc

(19) United States
(12) Patent Application Publication
(10) Pub. No.: US 2026/0078390 A1
(43) Pub. Date: Mar. 19, 2026

(54) Thph2 PROTEIN FROM TRICHODERMA HARZIANUM, ZmGLP1-17 PROTEIN FROM ZEA MAYS L., AND METHOD FOR PREPARING TRANSGENIC ZEA MAYS L.

(71) Applicants: SHANGHAI JIAO TONG UNIVERSITY, Shanghai (CN); Shanghai Dajing Bio-Engineering Co., Ltd., Shanghai (CN); HETAO COLLEGE, Bayannur (CN)

(72) Inventors: Jie CHEN, Shanghai (CN); Bo LANG, Shanghai (CN); Xifen ZHANG, Shanghai (CN); Yaqian LI, Shanghai (CN); Xinhua WANG, Shanghai (CN); Congzhou YANG, Shanghai (CN); Jing WANG, Shanghai (CN)

(73) Assignees: SHANGHAI JIAO TONG UNIVERSITY, Shanghai (CN); Shanghai Dajing Bio-Engineering Co., Ltd., Shanghai (CN); HETAO COLLEGE, Bayannur (CN)

(21) Appl. No.: 19/223,071

(22) Filed: May 30, 2025

(30) Foreign Application Priority Data
Sep. 19, 2024 (CN) 202411309083.2

(51) Int. Cl. C12N 15/82; A01N 63/38; A01P 3/00; C07K 14/415; C12N 9/42; C12P 19/34

(52) U.S. Cl. CPC C12N 15/8205; A01N 63/38; A01P 3/00; C07K 14/415; C12N 9/2437; C12N 15/8282; C12P 19/34; C12Y 302/01091

(57) ABSTRACT

Provided are a southern corn leaf blight-resistant Thph2 protein from Trichoderma harzianum, a ZmGLP1-17 protein from Zea mays L., and a method for preparing transgenic Zea mays L. Further provided are a cellobiohydrolase protein Thph2 from Trichoderma harzianum T30, a coding gene for the cellobiohydrolase protein, and use of the cellobiohydrolase protein in inducing a response of a GLP ZmGLP1-17 from Zea mays L. against southern corn leaf blight. Trichoderma harzianum strain OEthph2 overexpressing a Thph2 gene is constructed by a transgenic technique. OEthph2 may induce the expression of the GLP ZmGLP1-17 in roots of Zea mays L. and enhance the resistance of leaves of Zea mays L. to the infection of Cochliobolus heterostrophus. ZmGLP1-17 transgenic Zea mays L. material is constructed. The OEthph2 engineered strain may be combined with the ZmGLP1-17 transgenic Zea mays L. material to synergistically improve a control effect for southern corn leaf blight.

### CROSS REFERENCE TO THE RELATED APPLICATIONS

This application is based upon and claims priority to Chinese Patent Application No. 202411309083.2, filed on Sep. 19, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named WGJB0238-Track-One_Sequence_Listing.xml, created on May 7, 2025, and is 25,178 bytes in size.

### TECHNICAL FIELD

The present disclosure relates to the field of biological control. Specifically, the present disclosure relates to a Thph2 protein from Trichoderma harzianum, a ZmGLP1-17 protein from Zea mays L., and a method for preparing transgenic Zea mays L. The cellobiohydrolase protein Thph2 from Trichoderma harzianum may interact with the germin-like protein (GLP) ZmGLP1-17 from Zea mays L. to synergistically induce the systemic resistance of Zea mays L. and thus improve the resistance of leaves of Zea mays L. to southern corn leaf blight.

### BACKGROUND

Zea mays L. is one of the most important cash crops worldwide, and is also the leading grain crop in China. Southern corn leaf blight (Cochliobolus heterostrophus (C. heterostrophus) is an important disease affecting the safe corn production. In severe outbreak years, southern corn leaf blight may cause significant losses to corn production.

Trichoderma, a beneficial fungus ubiquitous in root ecosystems and soils, may not only inhibit the invasion of soil-borne pathogens and foster the growth of crops, but also induce the systemic acquired resistance in plants, thereby controlling the foliar diseases of crops. Studies have shown that Trichoderma may trigger the increase of reactive oxygen species (ROS) and calcium levels in leaves of Zea mays L. by interacting with the root system, thereby activating and enhancing the systemic immune defense response of Zea mays L from to leaf. Therefore, the treatment of seeds with Trichoderma as a biological control agent is a crucial technical approach for the eco-friendly control of southern corn leaf blight.

Previous studies have shown that cellulase produced by Trichoderma plays a significant role in the biological control of plant diseases. On the one hand, the cellulase may degrade the cell walls of pathogenic oomycetes (such as Pythium spp.) to promote the hyperparasitism of Trichoderma against these pathogens. On the other hand, the cellulase produced by Trichoderma and cellobiose resulting from cellulose degradation by the cellulase may induce the resistance of plants to fungal diseases (such as Curvularia lunata and Fusarium verticillioides) in leaves and cars. However, the mechanism of cellulase to induce the systemic resistance of Zea mays L. is currently not clear.

### SUMMARY

The present disclosure is intended to provide a cellobiohydrolase protein Thph2 from Trichoderma harzianum that may control southern corn leaf blight, a coding gene for the cellobiohydrolase protein Thph2, a specifically-responsive Zea mays L. root-specific target protein ZmGLP1-17, and a use technology. Accordingly, the present disclosure further develops a novel biological control technique for enhancing systemic resistance of Zea mays L. Specific technical solutions are as follows:

The present disclosure provides a cellobiohydrolase protein Thph2 from Trichoderma harzianum, where the cellobiohydrolase protein Thph2 is the following protein in (S1) or (S2): (S1) a protein with an amino acid sequence set forth in SEQ ID NO: 1; and

(S2) a derived protein that is produced through substitution, deletion, or addition of 1 to 10 amino acid residues based on the amino acid sequence set forth in SEQ ID NO: 1 and has a same function as the protein described in the (1).

The cellobiohydrolase protein Thph2 includes a distinct cellulose-binding conserved domain at an N terminus. The clustering analysis results show that the cellobiohydrolase protein Thph2 exhibits a maximum homology (a similarity of 85.7%) with a CBH2 protein derived from Trichoderma reesei (T. reesei).

According to a specific embodiment, the Trichoderma harzianum is Trichoderma harzianum T30, which was deposited in China General Microbiological Culture Collection Center (CGMCC) in Institute of Microbiology, Chinese Academy of Sciences at NO. 1 West Beichen Road, Chaoyang District, Beijing on Jun. 15, 2021, with an accession number of CGMCC 22479.

The present disclosure also provides a coding gene for the cellobiohydrolase protein Thph2.

As an example, the coding gene for the cellobiohydrolase protein Thph2 has a nucleotide sequence selected from the group consisting of the following (X1) to (X4):
(X1) a nucleotide sequence set forth in SEQ ID NO: 2;
(X2) a polynucleotide encoding the protein with the amino acid sequence set forth in SEQ ID NO: 1;

(X3) a DNA sequence that has a homology of 90% or more with the nucleotide sequence set forth in SEQ ID NO: 2 and encodes a protein with a same function as a protein encoded by the nucleotide sequence set forth in SEQ ID NO: 2; and

(X4) a nucleotide sequence produced through hybridization with the nucleotide sequence set forth in SEQ ID NO: 2 under stringent conditions, where the stringent conditions are as follows: the hybridization is conducted in a 0.1× saline-sodium phosphate-EDTA (SSPE) solution including 0.1% of sodium dodecyl sulfate (SDS) or a 0.1× saline-sodium citrate (SSC) solution including 0.1% of SDS at 65° C., and membrane washing is conducted in a same solution as the hybridization is conducted.

The present disclosure also provides a transformant OEthph2 overexpressing a cellobiohydrolase protein Thph2 from Trichoderma harzianum T30.

The present disclosure has discovered that leaves of Zea mays L. inoculated with OEthph2 exhibit significantly-better southern corn leaf blight resistance than leaves of Zea mays L. inoculated with T30.

According to a specific embodiment, a role of Thph2 in inducing the systemic resistance of Zea mays L. is verified by applying 50 mL of a spore suspension of a Trichoderma Thph2 transformant OEthph2 (at a concentration of 1× 10⁶ cfu/mL) to roots of each Zea mays L. plant and inoculating a C. heterostrophus spore suspension into leaves of Zea mays L. Results show that disease spots on leaves of Zea mays L. inoculated with the Trichoderma Thph2 transformant OEthph2 are significantly reduced (FIGS. 2A-2D), the expression of related defense response genes is enhanced (FIGS. 3A-3B), and the expression level of ZmGLP1-17 increases (FIG. 4), indicating that the cellobiohydrolase protein Thph2 from Trichoderma harzianum plays a positive regulatory role in inducing the resistance of Zea mays L.

According to a specific embodiment, the present disclosure achieves the overexpression of a Thph2 gene in Trichoderma through Agrobacterium-mediated transformation to produce a transgenic Trichoderma transformant OEthph2. Test results show that an expression level of cellobiohydrolase in the transgenic transformant is significantly higher than an expression level of cellobiohydrolase in a wild type (FIG. 1). It is inferred accordingly that the overexpression of Thph2 can be achieved by a transgenic technique to produce a Trichoderma engineered strain resource that has a high expression level of cellobiohydrolase and can induce the resistance of Zea mays L. to southern corn leaf blight.

The present disclosure also provides GLP ZmGLP1-17 from Zea mays L., where the GLP ZmGLP1-17 is the following protein in (Y1) or (Y2):
(Y1) a protein with an amino acid sequence set forth in SEQ ID NO: 3; and
(Y2) a derived protein that is produced through substitution, deletion, or addition of 1 to 10 amino acid residues based on the amino acid sequence set forth in SEQ ID NO: 3 and has a same function as the protein described in the (1).

The protein ZmGLPs is a stable hydrophilic protein, which includes a cupin_OxOx conserved domain, belongs to the Cupin superfamily of proteins, and has a transmembrane domain at an N terminus and a signal peptide cleavage site located between the 22nd and 23rd amino acids. Since plant GLPs typically have superoxide dismutase (SOD) and oxalate oxidase (OXO) activities, a content of H₂O₂ may increase.

According to a specific embodiment, the protein ZmGLP1-17 is produced through a yeast two-hybrid experiment involving the protein Thph2 and a Zea mays L. root cDNA library. Moreover, an interaction between the protein ZmGLP1-17 and the protein Thph2 in the resistance to southern corn leaf blight is verified by methods such as bimolecular fluorescence complementation (BiFC) and co-immunoprecipitation (Co-IP) (FIGS. 5A-5C).

The present disclosure also provides a coding gene for the GLP ZmGLP1-17.

According to a specific embodiment, the coding gene has a nucleotide sequence selected from the group consisting of the following (SS1) to (SS4):
(SS1) a nucleotide sequence set forth in SEQ ID NO: 4;
(SS2) a polynucleotide encoding the protein with the amino acid sequence set forth in SEQ ID NO: 3;
(SS3) a DNA sequence that has a homology of 90% or more with the nucleotide sequence set forth in SEQ ID NO: 4 and encodes a protein with a same function as a protein encoded by the nucleotide sequence set forth in SEQ ID NO: 4; and
(SS4) a nucleotide sequence produced through hybridization with the nucleotide sequence set forth in SEQ ID NO: 4 under stringent conditions, wherein the stringent conditions are as follows: the hybridization is conducted in a 0.1×SSPE solution comprising 0.1% of SDS or a 0.1×SSC solution comprising 0.1% of SDS at 65° C., and membrane washing is conducted in a same solution as the hybridization is conducted.

Since the activation of a Zea mays L. root immune system by the Thph2 protein from Trichoderma harzianum serves as the foundation for inducing a defense response in leaves, the development of a technology for inducing the resistance in Zea mays L. with the Thph2 protein from Trichoderma harzianum and the construction of transgenic Zea mays L. that expresses a protein ZmGLP1-17 specifically interacting with Thph2 can provide key technologies for inducing the resistance of Zea mays L. to southern corn leaf blight, which is systemically induced by a Trichoderma harzianum.

The present disclosure also provides a method for cultivating transgenic Zea mays L. overexpressing GLP ZmGLP1-17 from Zea mays L., including the following steps: (1) cloning a coding gene for the GLP ZmGLP1-17, and introducing the coding gene into Agrobacterium to produce a recombinant strain; and
(2) constructing the transgenic Zea mays L. overexpressing ZmGLP1-17 through Agrobacterium-mediated plant genetic transformation.

As an example, a specific process for cloning the coding gene for the GLP ZmGLP1-17 in the step (1) includes: synthesizing a primer pair to amplify a full-length fragment or any fragment of the coding gene for the GLP ZmGLP1-17; and cloning the coding gene for the GLP ZmGLP1-17 with the primer pair through polymerase chain reaction (PCR), where the primer pair includes a forward primer and a reverse primer; and the forward primer has a nucleotide sequence set forth in SEQ ID NO: 11 and the reverse primer has a nucleotide sequence set forth in SEQ ID NO: 12.

The present disclosure also provides a method for synergistically controlling southern corn leaf blight with a cellobiohydrolase protein Thph2 from Trichoderma harzianum and transgenic Zea mays L. overexpressing GLP ZmGLP1-17 from Zea mays L., including the following steps:
(1) preparing Trichoderma harzianum T30 or a Thph2-overexpressing transformant into a spore suspension; and
(2) applying the spore suspension to transgenic Zea mays L. overexpressing a protein ZmGLP1-17 prepared by the method described above through root drenching.

As an example, the spore suspension applied to the transgenic Zea mays L. has a concentration of 10⁶ cfu/mL, and is applied in a volume of 50 mL/plant.

The GLP ZmGLP1-17 from Zea mays L. plays a positive regulatory role in the resistance to southern corn leaf blight. The transgenic Zea mays L. overexpressing ZmGLP1-17 exhibits significantly-higher resistance to southern corn leaf blight than non-transgenic Zea mays L.

Compared with the prior art, the present disclosure has the following beneficial effects: The present disclosure acquires a cellobiohydrolase protein Thph2 with a function of inducing the resistance of Zea mays L. to southern corn leaf blight by Trichoderma harzianum, and a coding gene for the cellobiohydrolase protein, which provides a new genetic resource as a biological pesticide for the biological control of southern corn leaf blight. In addition, a coding gene for a ZmGLP1-17 protein is isolated from Zea mays L. and cloned, where the ZmGLP1-17 protein may specifically interact with the cellobiohydrolase protein Thph2 from Trichoderma harzianum to induce the resistance of Zea mays L. to southern leaf blight in corn. The coding gene for the ZmGLP1-17 protein is introduced into Zea mays L. through Agrobacterium-mediated transformation to produce a transgenic Zea mays L. material with resistance to southern corn leaf blight. The combination of a cellobiohydrolase Thph2-overexpressing Trichoderma harzianum engineered strain with a disease-resistant Zea mays L. GLP ZmGLP1-17-transgenic material may provide a novel key technology for the biological control of southern corn leaf blight.

### BRIEF DESCRIPTION OF THE DRAWINGS

FIG. 1 shows the expression of Thph2 in a transformant (OEthph2) overexpressing a cellobiohydrolase Thph2 from Trichoderma harzianum;

FIGS. 2A-2D show the phenotypic detection results for the induction of resistance of Zea mays L. to southern corn leaf blight by T30 and OEthph2 strains, including phenotypic images (FIG. 2A) and data analysis (FIG. 2B) for disease spots on Zea mays L. leaves in vitro inoculated with the different strains; and phenotypic images (FIG. 2C) and data analysis (FIG. 2D) for disease lesion on Zea mays L. leaves in vivo inoculated with the different strains;

FIGS. 3A-3B show the detection results of related defense response genes during the induction of resistance of Zea mays L. to southern corn leaf blight by T30 and OEthph2 strains, where (FIG. 3A) shows the expression of hpl, aos, lox5, and opr1 genes at 36 h after the inoculation of C. heterostrophus and (FIG. 3B) shows the expression of zmpr1, zmpr5, and pal genes at 36 h after the inoculation of C. heterostrophus;

FIG. 4 shows the expression of ZmGLP in roots and leaves after the inoculation of T30 and OEthph2 into roots;

FIGS. 5A-5C show the screening and detection of Thph2-interacting proteins, where (FIG. 5A) shows Thph2-interacting proteins screened by Y2H; (FIG. 5B) shows the verification of an interaction between Thph2 and ZmGLP by BiFC; and (FIG. 5C) shows the verification of an interaction between Thph2 and ZmGLP by Co-IP;

FIGS. 6A-6B are schematic diagrams for the construction and detection of a knockout vector for Zea mays L.;

FIGS. 7A-7C show the detection results of basic physiological indexes of B104, Δglp, and oeglp and the detection results of disease resistance phenotypes; and

FIGS. 8A-8B show the phenotypic images for B104, Δglp1, and oeglp1 after roots are treated with ddH2O and T30 and OEthph2 spore suspensions for 7 d and leaves are inoculated with C. heterostrophus in (FIG. 8A) and the measurement results of disease spot areas on leaves of Zea mays L. in (FIG. 8B).

### DETAILED DESCRIPTION OF THE EMBODIMENTS

The following examples are intended to illustrate the present disclosure, but not to limit the scope of the present disclosure. The methods adopted in the following examples all are the conventional methods unless otherwise specified. All primers are synthesized by Shanghai Saiheng Biotechnology Co., Ltd. The sequencing is conducted by Shanghai Saiheng Biotechnology Co., Ltd. The restriction endonuclease, ligase, T1 vector, DNA marker, Tag DNA polymerase, dNTP, etc. adopted in the experiments are purchased from Takara and Vazyme. The reverse transcription kit is purchased from Vazyme. The plasmid extraction kit, gel extraction kit, and genome extraction kit all are purchased from Vazyme, and the methods all are conducted according to the instructions.

All quantitative tests in the following examples are set to run in triplicate, and the results are averaged.

The biological materials adopted in the present disclosure are as follows: Trichoderma harzianum T30, which is taxonomically classified under the Trichoderma, Hypocreaceae, Hypocreales, Sordariomycetes, and Ascomycota, and is deposited at the Key Laboratory of Urban Agriculture (South) of the Ministry of Agriculture, School of Agriculture and Biology, Shanghai Jiao Tong University; and a Zea mays L. inbred line B104, purchased from WIMI Biotech.

### Example 1 Cloning of a Trichoderma Cellobiohydrolase Gene and Construction of a Transformant

1. Since the Trichoderma transcription factor The6, a zinc finger protein, functioned by regulating Thph2, a positive clone plasmid interacting with The6 was produced through yeast one-hybrid assay, and sequencing was conducted to obtain a DNA sequence. Primers were designed with the DNA sequence as a template. Flanking DNA sequences of the known sequence were then amplified through inverse PCR. Through 5 rounds of PCR in total, about 6 kb of a DNA sequence of the gene was amplified as a whole. Then a cDNA fragment of the gene was acquired through analysis and prediction, and a full-length fragment of the gene was amplified through 5′ and 3′ Rapid Amplification of cDNA Ends (RACE).

2. To construct an overexpression vector for Thph2, a reading frame fragment (SEQ ID NO: 2) of the cDNA of the gene was selected as an expressing sequence for the overexpression vector. A TrpC promoter sequence (SEQ ID NO: 13) and a TrpC terminator sequence (SEQ ID NO: 14) from Aspergillus were adopted as leader and terminator sequences for the target expressing gene, respectively. The primer sequences were designed as follows: primers A1 (SEQ ID NO: 5) and A2 (SEQ ID NO: 6) were used to amplify the TrpC promoter fragment, primers B1 (SEQ ID NO: 7) and B2 (SEQ ID NO: 8) were used to amplify the Thph2 fragment, and primers C1 (SEQ ID NO: 9) and C2 (SEQ ID NO: 10) were used to amplify the TrpC terminator fragment. The fragments were ligated to a digested vector with the ClonExpress IIOne Step Cloning Kit (Vazyme) to produce an overexpression cassette for the gene. The overexpression cassette was transformed into Escherichia coli DH5a, then single colonies were picked, and the plasmid was extracted and then transformed into Agrobacterium AGL1. The overexpression cassette was transformed into Trichoderma harzianum T30 through Agrobacterium tumefaciens-mediated transformation (ATMT) to produce a transformant OEthph2.

3. After being stably inherited, the transformant was transferred to a PDA plate and cultured at 28° C. for 7 d. DNA was extracted according to the instructions of the FastPure Plant DNA Isolation Mini Kit (Vazyme). With an extraction product as a template, PCR amplification was conducted using primers set forth in SEQ ID NO: 15 and SEQ ID NO: 16. An amplification product was a resistant marker gene G418. The amplification result indicated the successful transformation. The total RNA was extracted from cells by a Trizol method. The integrity of RNA was identified by formaldehyde-denaturing gel electrophoresis. Then a purity and concentration of RNA were determined on a spectrophotometer (Thermo Scientific NANODROP 2000Spectrophotometer). The reverse transcription was conducted with the PrimeScript™ 1st Strand cDNA Synthesis Kit from Takara to produce a cDNA template.

4. Identification of Thph2 expression: Primers for real-time PCR were designed with the oligo7 software. Specific primer sequences were as follows:

SEQ ID NO: 17 (Thph2-F):
5′-GTTCTCTGCCATCGCTCTCT-3′;
and
SEQ ID NO: 18 (Thph2-R):
5′-AGCGCCACAGCACTTGGGGA-3′.

An internal reference gene for Trichoderma was Actin. Primers for Actin were as follows:

SEQ ID NO: 19 (Actin-F):
5′-GTATCATGATCGGTATGGGTCAGA-3′;
and
SEQ ID NO: 20 (Actin-R):
5′-TAGAAGGTGTGGTGCCAGATCTT-3′.

With a first strand of the synthesized cDNA as a template, the amplification was conducted using specific primers for the target gene and the internal reference gene to allow the fluorescence quantitative analysis. The real-time PCR was conducted on a Roche96 real-time quantitative PCR instrument, with a reaction system of 20 μL. The real-time PCR was conducted by a three-step method: denaturation at 95° C. for 30 s, and 40 cycles of 95° C. for 20 s, 55° C. for 20 s, and 72° C. for 20 s. After each amplification was completed, a melt curve was plotted to verify whether an amplification product was specifically produced.

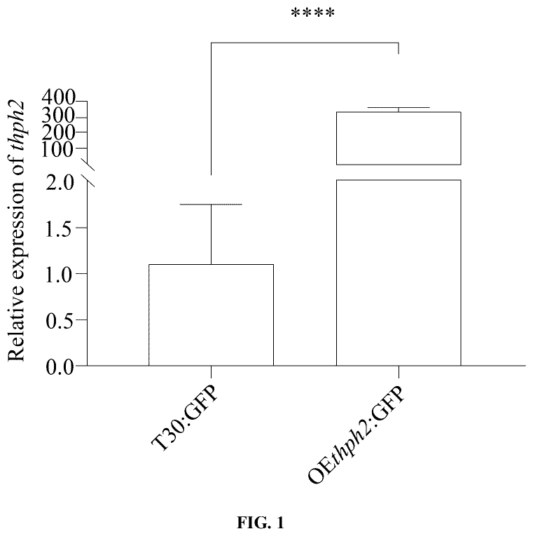

The relative quantitative analysis was conducted by the 2^{-ΔΔCt} method. Results were shown in FIG. 1. An expression level of Thph2 in the transformant OEthph2 was significantly higher than an expression level of Thph2 in the wild-type strain T30, which provided a basis for the subsequent experiments.

### Example 2 Use and Functional Detection of a Transformant Overexpressing a Trichoderma Cellobiohydrolase Gene

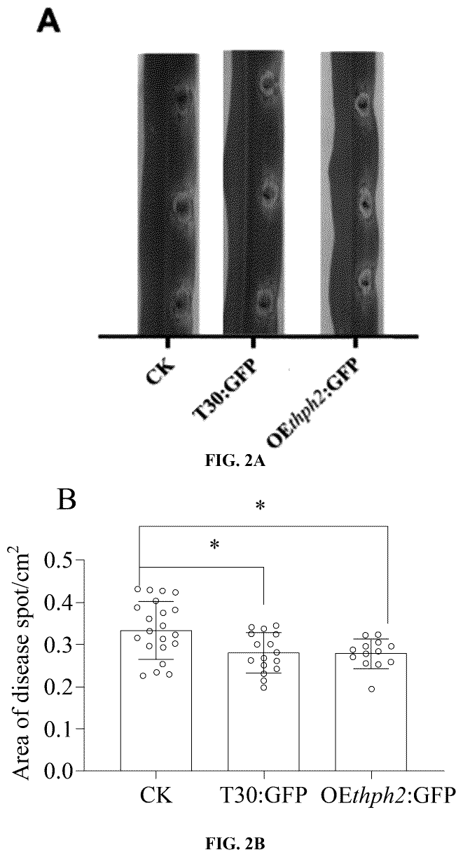

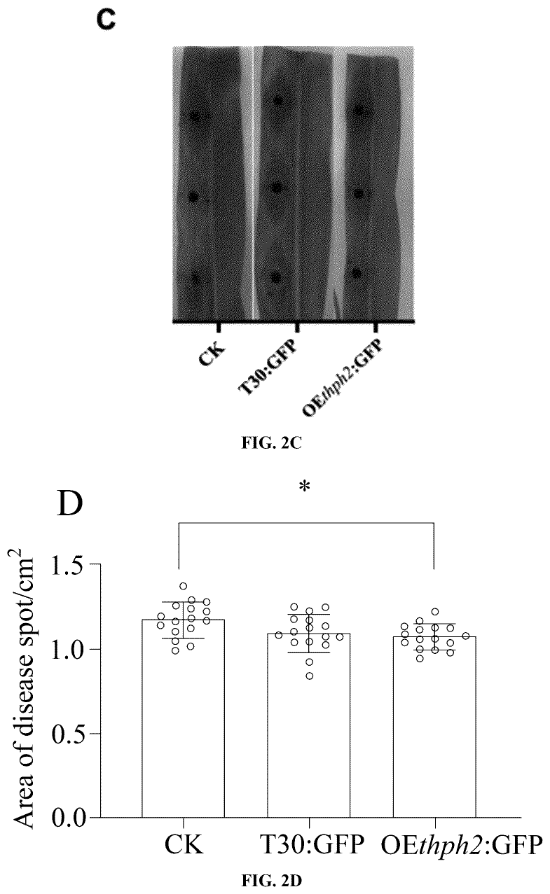

1. To verify an ability of wild-type Trichoderma and the overexpression transformant to induce the resistance of Zea mays L. through a root treatment, the effects of the T30 and overexpression transformant OEthph2 on the resistance of leaves of Zea mays L. to southern corn leaf blight (C. heterostrophus) were compared. A spore suspension of T30 or OEthph2 (at a concentration of 1× 10⁶ cfu/mL) was applied to roots of Zea mays L. in a volume of 50 mL/plant. After roots were treated with Trichoderma (T30 or OEthph2) for 7 d, leaves were inoculated with C. heterostrophus for 72 h, and a disease spot size on leaves was measured for defense assessment. In this example, the following two methods were adopted for inoculating leaves with C. heterostrophus: in vitro leaf inoculation and in vivo leaf inoculation. Leaves of plants whose roots were not inoculated with Trichoderma developed large disease spots early, while leaves of plants treated with the T30 or OEthph2 strain developed very small disease spots later. Moreover, disease spots in OEthph2-treated plants were smaller than disease spots in T30-treated plants, as shown in FIGS. 2A-2D.

A preparation process of the Trichoderma spore suspension was as follows: The T30 wild-type strain and the overexpression transformant OEthph2 each were inoculated into a PDA medium and cultured at 28° C. for 7 d, and the Trichoderma spore suspension was prepared. A preparation process of the PDA medium was as follows: 200 g of a potato was taken, peeled, diced, and then steamed. A resulting supernatant was taken, and 20 g of glucose and 20 g of an agar powder were added to produce a mixture. The mixture was diluted with deionized water to 1 L, dispensed in 250 mL erlenmeyer flasks, and autoclaved at 121° C. for 30 min.

2. To confirm the sustainable effects of T30 and OEthph2 for inducing the resistance of Zea mays L. to southern corn leaf blight, the expression of four JA pathway-related defense genes in Zea mays L. was detected. Marker genes of the JA pathway were lox5, aos, hpl, and opr1.

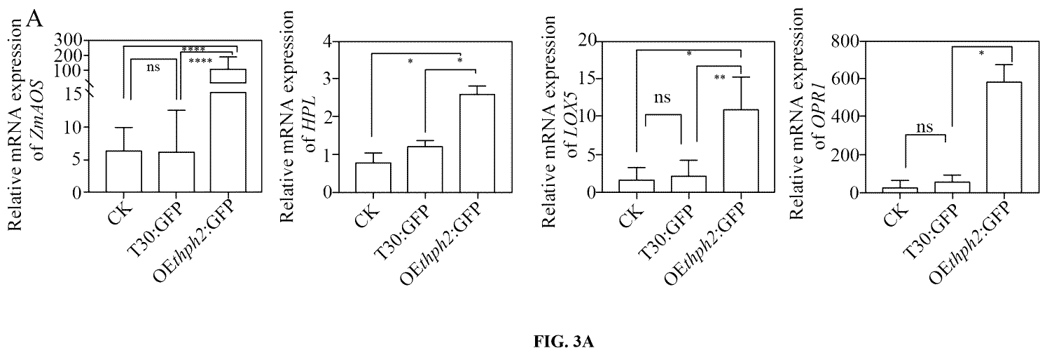

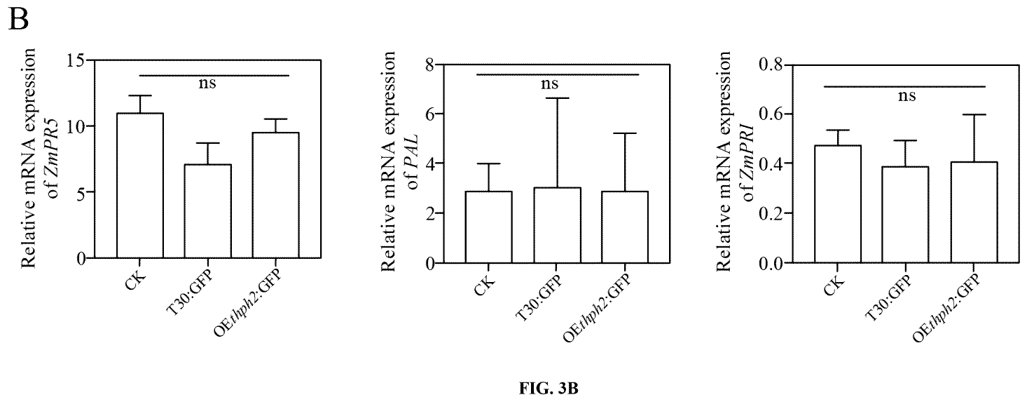

3. Results showed that, after roots of Zea mays L. were colonizied with OEthph2, the expression of aos was significantly up-regulated, there was little difference between T30-treated plants and CK in terms of the expression of aos, and the expression of lox5 was similar to the expression of AOS. The expression levels of Hpl and OPRI in leaves of Zea mays L. inoculated with OEthph2 were higher than the expression levels of hpl and opr1 in leaves of Zea mays L. inoculated with T30 (FIG. 3A).

### Example 3 Construction and Resistance Evaluation for Transgenic Zea mays L. Overexpressing a GLP Gene ZmGLP1-17 from Zea mays L.

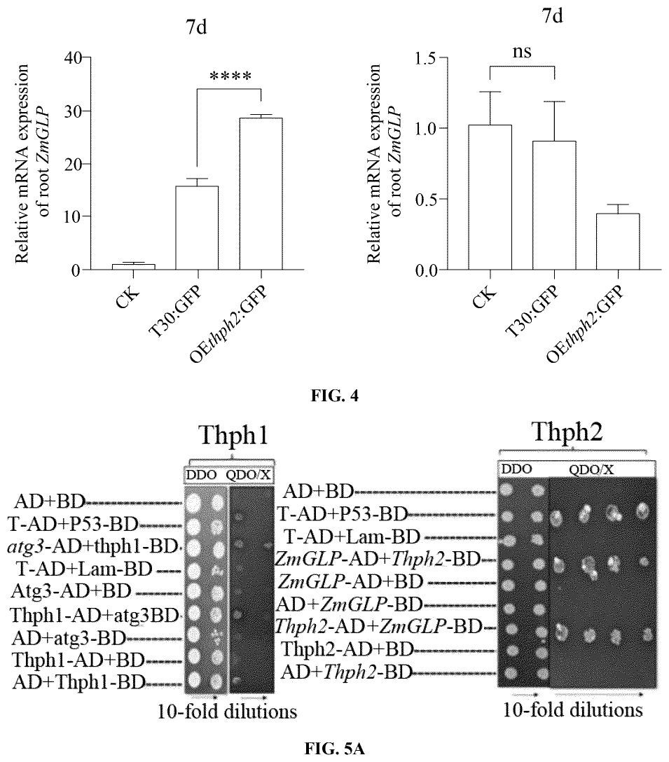

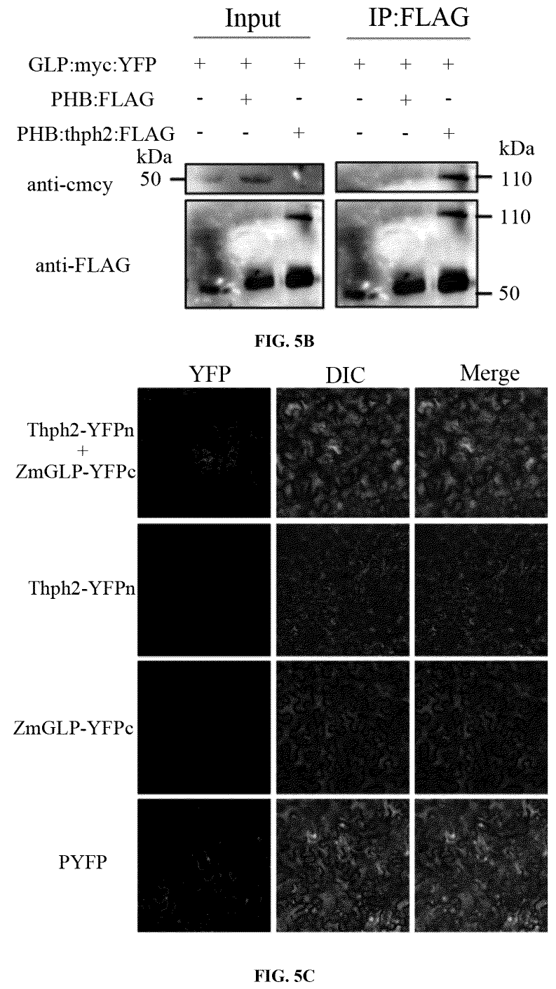

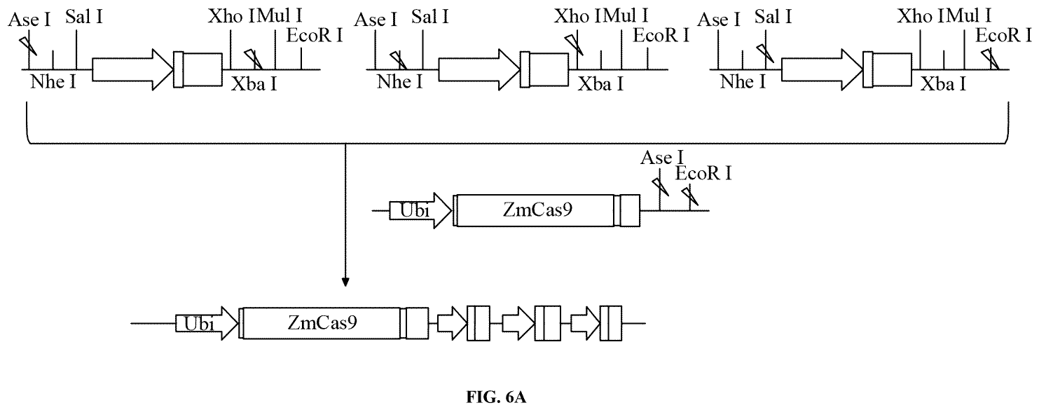

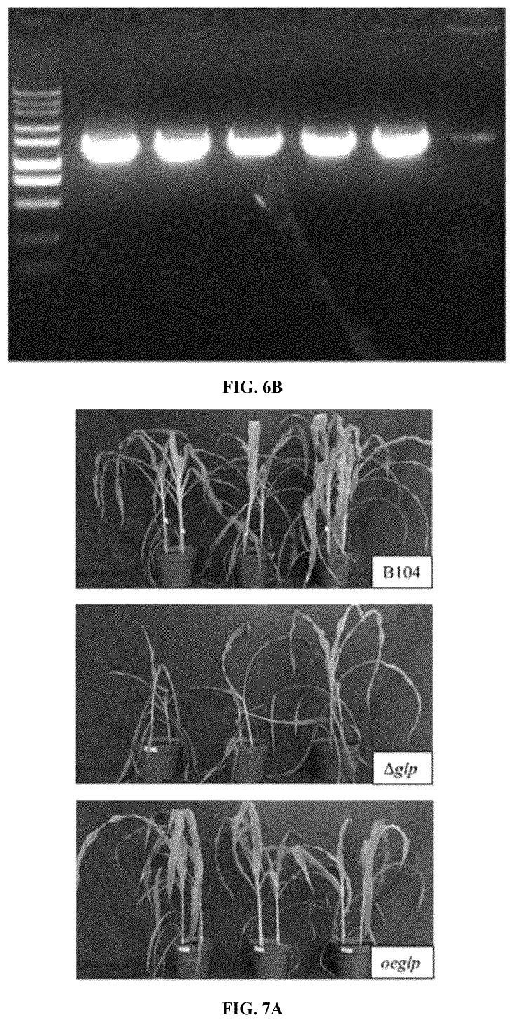

1. An interaction between Thph2 and ZmGLP1-17 was verified. The interaction between Thph2 and ZmGLP1-17 was verified by BiFC and Co-IP (FIGS. 5A-5C). An expression level of ZmGLP1-17 in Zea mays L. treated with the OEthph2 engineered strain was significantly higher than an expression level of ZmGLP1-17 in Zea mays L. treated with the wild-type strain T30 (FIG. 4).

2. A full-length coding gene for ZmGLP1-17 was amplified using SEQ ID NO: 21 and SEQ ID NO: 22. A ZmGLP1-17 overexpression vector and a ZmGLP1-17 knockout vector were constructed separately and then introduced into a recombinant plasmid Agrobacterium. Zea mays L. immature embryos were transformed with a transformed recombinant plasmid Agrobacterium and cultivated to produce transgenic Zea mays L. plants overexpressing ZmGLP1-17 (namely, a ZmGLP1-17-overexpressing Zea mays L. line OEglp, referred to as OEglp hereafter) and ZmGLP1-17-knockout Zea mays L. plants Δglp (referred to as Δglp hereafter). PCR validation was conducted.

3. Seeds of homozygous T2 generations of the ZmGLP1-17-overexpressing Zea mays L. line OEglp, the ZmGLP1-17-knockout Zea mays L. line Δglp, and a control line (namely B104) were placed in petri dishes and soaked in deionized water to allow imbibition and germination. When seedlings grew to a height of about 5 cm, seedlings with a consistent growth status were selected from each line and transferred to plastic pots each including a same weight of a soil mixture (nutrient soil:vermiculite=1:1). Each pot was planted with 15 tested positive seedlings. Three replicates were set for each line. The seedlings were cultivated in a sunlight greenhouse for 10 d, and then inoculated for disease resistance. A spore suspension of C. heterostrophus was prepared at a concentration of 1×10⁵ cfu/mL and inoculated on leaves of Zea mays L., where each leaf was inoculated with 3 drops of the spore suspension, and each drop was of 10 μL. After the inoculation, a high-temperature and high-humidity environment needed to be maintained for 24 h. Phenotypic changes in transgenic and wild-type Zea mays L. were observed, and the disease progression was recorded. Results intuitively demonstrated that the overexpression of the gene ZmGLP1-17 played a positive regulatory role in the resistance of plants to southern corn leaf blight.

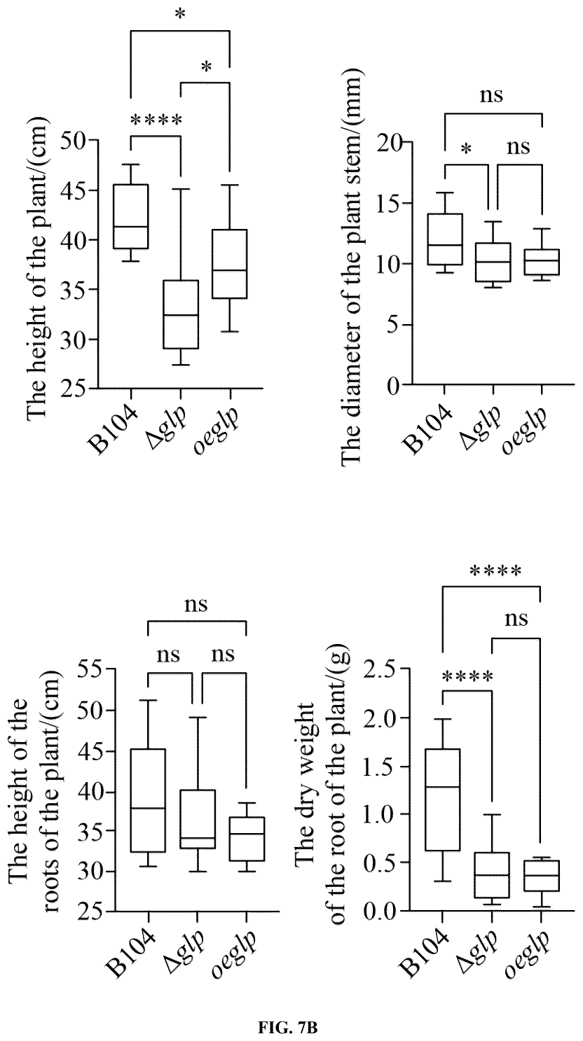

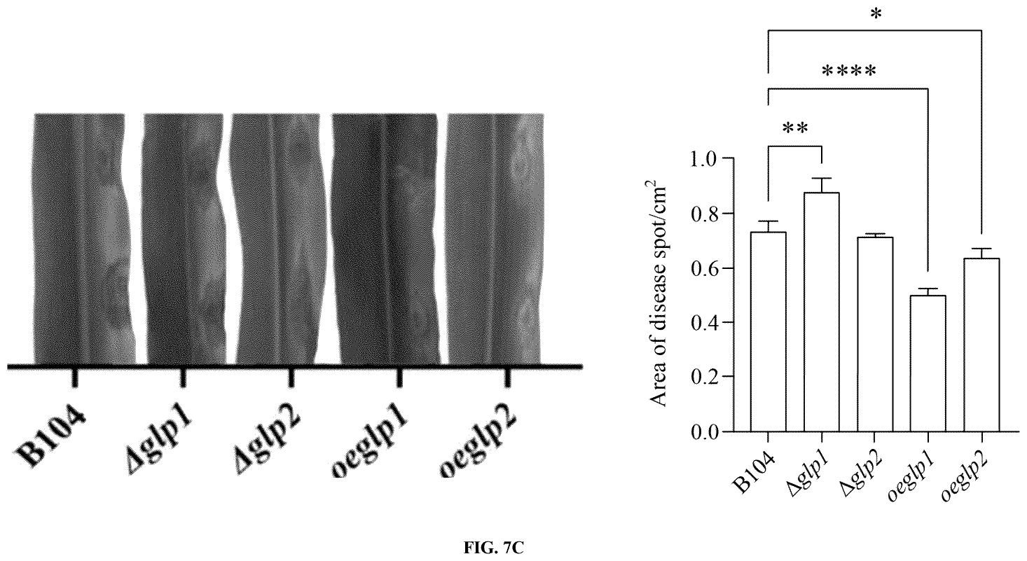

4. Result confirmation: In this experiment, two ZmGLP1-17-knockout plants Δglp1 and Δglp2 and two ZmGLP1-17-overexpressing plants OEglp1 and OEglp2 were selected and subjected to fluorescence quantitative detection. Detection results confirmed the successful knockout and overexpression. It could be determined by measuring disease spot sizes that, after the knockout of ZmGLP1-17, the resistance of Zea mays L. to southern corn leaf blight was weakened, there was a significant difference between the Δglp1 plant and the wild-type plant, and Δglp2 had a slightly-larger disease spot area than the wild-type plant. After the overexpression of ZmGLP1-17, the resistance of Zea mays L. to southern corn leaf blight was enhanced, and both oeglp1 and oeglp2 had a significant difference from the wild-type plant (FIG. 7C).

### Example 4 Synergistic Control of Southern Corn Leaf Blight by a Trichoderma Cellobiohydrolase-Overexpressing Engineered Strain and GLP-Transgenic Zea mays L.

1. Δglp1 with a significant knockout phenotype and OEglp1 with a significant overexpression phenotype were selected for the subsequent experiment.

2. Spores of a wild-type Trichoderma harzianum strain (T30) and an overexpression strain (OEthph2) each were prepared into a spore suspension at a concentration of 10⁶ cfu/mL.

3. A Trichoderma spore suspension was applied to roots of B104, Δglp1, and OEglp1 through root drenching at 50 mL/plant. 6 treatments were set in total, and 6 plants were selected for each treatment.

4. After roots were treated with Trichoderma for 7 d, leaves were inoculated with C. heterostrophus for 72 h, and a disease spot size on leaves was measured for defense assessment.

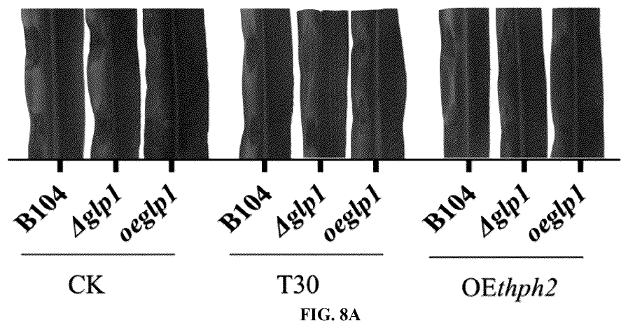

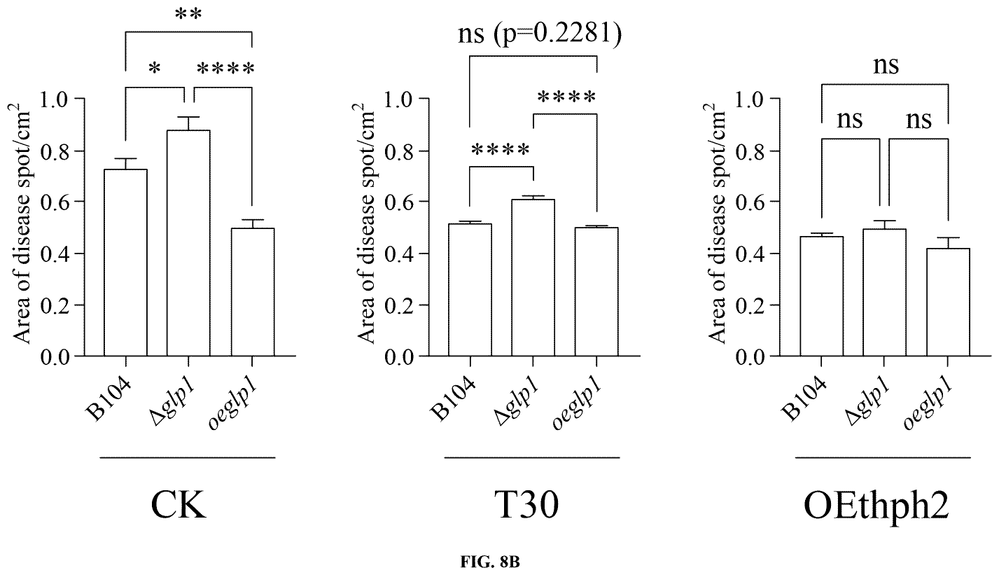

5. Results: After a spore suspension of T30 or OEthph2 was applied to roots, disease spot sizes in all treatments were reduced compared to a control group (FIG. 8A), but the overall trend remained unchanged, with disease spot areas in following the order: Δglp1>B104>oeglp1 (FIG. 8B). To investigate the synergistic role of Trichoderma harzianum Thph2 and Zea mays L. ZmGLP1-17 in enhancing the resistance of Zea mays L. to southern corn leaf blight, the different treatments were subjected to comparative analysis. A disease spot area of oeglp1/CK was larger than a disease spot area of oeglp1/T30, and was also larger than a disease spot area of oeglp1/OEthph2, indicating that Trichoderma harzianum Thph2 and Zea mays L. ZmGLP1-17 played a synergistic role in enhancing the resistance of Zea mays L. to southern corn leaf blight. The resistance in Zea mays L. treated with root drenching of the OEthph2-overexpressing strain was generally enhanced, and the resistance was also increased in Δglp1. It indicated that Trichoderma Thph2 may have other action targets in the roots of Zea mays L., and also play a role in inducing the resistance.

What is claimed is:

1. A method for cultivating transgenic Zea mays L. overexpressing GLP ZmGLP1-17 from Zea mays L., comprising the following steps:
(1) cloning a coding gene for the GLP ZmGLP1-17, and introducing the coding gene into Agrobacterium to produce a recombinant strain; and
(2) constructing the transgenic Zea mays L. overexpressing the GLP ZmGLP1-17 through Agrobacterium-mediated plant genetic transformation.

2. The method according to claim 1, wherein a process for cloning the coding gene for the GLP ZmGLP1-17 in the step (1) comprises: synthesizing a primer pair to amplify a full-length fragment or any fragment of the coding gene for the GLP ZmGLP1-17; and cloning the coding gene for the GLP ZmGLP1-17 with the primer pair through polymerase chain reaction (PCR), wherein the primer pair comprises a forward primer and a reverse primer; and the forward primer has the nucleotide sequence set forth in SEQ ID NO: 11 and the reverse primer has the nucleotide sequence set forth in SEQ ID NO: 12;
wherein the coding gene for the GLP ZmGLP1-17 has a nucleotide sequence selected from the group consisting of the following (SS1) to (SS4):
(SS1) the nucleotide sequence set forth in SEQ ID NO: 4;
(SS2) a polynucleotide encoding a protein with the amino acid sequence set forth in SEQ ID NO: 3;
(SS3) a DNA sequence having a homology of 90% or more with the nucleotide sequence set forth in SEQ ID NO: 4 and encoding a protein with a same function as a protein encoded by the nucleotide sequence set forth in SEQ ID NO: 4; and
(SS4) a nucleotide sequence produced through hybridization with the nucleotide sequence set forth in SEQ ID NO: 4 under stringent conditions, wherein the stringent conditions are as follows: the hybridization is conducted in a 0.1× saline-sodium phosphate-ethylene diamine tetraacetic acid (SSPE) solution comprising 0.1% of sodium dodecyl sulfate (SDS) or a 0.1× saline-sodium citrate (SSC) solution comprising 0.1% of SDS at 65° C., and membrane washing is conducted in a same solution as the hybridization is conducted.

3. A method for synergistically controlling southern corn leaf blight with a cellobiohydrolase protein Thph2 from Trichoderma harzianum and transgenic Zea mays L. overexpressing GLP ZmGLP1-17 from Zea mays L., comprising the following steps:
(1) preparing Trichoderma harzianum T30 or a Thph2-overexpressing transformant into a spore suspension; and
(2) applying the spore suspension to the transgenic Zea mays L. prepared by the method according to claim 1 through root drenching.

4. The method according to claim 1, wherein the GLP ZmGLP1-17 is the following protein in (Y1) or (Y2):
(Y1) a protein with the amino acid sequence set forth in SEQ ID NO: 3; and
(Y2) a derived protein produced through substitution, deletion, or addition of 1 to 10 amino acid residues based on the amino acid sequence set forth in SEQ ID NO: 3 and having a same function as the protein described in the (Y1).

5. The method according to claim 1, wherein the coding gene for the GLP ZmGLP1-17 has a nucleotide sequence selected from the group consisting of the following (SS1) to (SS4):
(SS1) the nucleotide sequence set forth in SEQ ID NO: 4;
(SS2) a polynucleotide encoding a protein with the amino acid sequence set forth in SEQ ID NO: 3;
(SS3) a DNA sequence having a homology of 90% or more with the nucleotide sequence set forth in SEQ ID NO: 4 and encoding a protein with a same function as a protein encoded by the nucleotide sequence set forth in SEQ ID NO: 4; and
(SS4) a nucleotide sequence produced through hybridization with the nucleotide sequence set forth in SEQ ID NO: 4 under stringent conditions, wherein the stringent conditions are as follows: the hybridization is conducted in a 0.1×SSPE solution comprising 0.1% of SDS or a 0.1×SSC solution comprising 0.1% of SDS at 65° C., and membrane washing is conducted in a same solution as the hybridization is conducted.

6. The method according to claim 3, wherein a process for cloning the coding gene for the GLP ZmGLP1-17 in the step (1) comprises: synthesizing a primer pair to amplify a full-length fragment or any fragment of the coding gene for the GLP ZmGLP1-17; and cloning the coding gene for the GLP ZmGLP1-17 with the primer pair through polymerase chain reaction (PCR), wherein the primer pair comprises a forward primer and a reverse primer; and the forward primer has the nucleotide sequence set forth in SEQ ID NO: 11 and the reverse primer has the nucleotide sequence set forth in SEQ ID NO: 12;
wherein the coding gene for the GLP ZmGLP1-17 has a nucleotide sequence selected from the group consisting of the following (SS1) to (SS4):
(SS1) the nucleotide sequence set forth in SEQ ID NO: 4;
(SS2) a polynucleotide encoding a protein with the amino acid sequence set forth in SEQ ID NO: 3;
(SS3) a DNA sequence having a homology of 90% or more with the nucleotide sequence set forth in SEQ ID NO: 4 and encoding a protein with a same function as a protein encoded by the nucleotide sequence set forth in SEQ ID NO: 4; and
(SS4) a nucleotide sequence produced through hybridization with the nucleotide sequence set forth in SEQ ID NO: 4 under stringent conditions, wherein the stringent conditions are as follows: the hybridization is conducted in a 0.1×SSPE solution comprising 0.1% of SDS or a 0.1×SSC solution comprising 0.1% of SDS at 65° C., and membrane washing is conducted in a same solution as the hybridization is conducted.

7. The method according to claim 3, wherein the cellobiohydrolase protein Thph2 is the following protein in (S1) or (S2):
(S1) a protein with the amino acid sequence set forth in SEQ ID NO: 1; and
(S2) a derived protein produced through substitution, deletion, or addition of 1 to 10 amino acid residues based on the amino acid sequence set forth in SEQ ID NO: 1 and having a same function as the protein described in the (S1).

8. The method according to claim 7, wherein the Trichoderma harzianum is the Trichoderma harzianum T30, the Trichoderma harzianum T30 is deposited in China General Microbiological Culture Collection Center (CGMCC) in Institute of Microbiology, Chinese Academy of Sciences at NO. 1 West Beichen Road, Chaoyang District, Beijing on Jun. 15, 2021, and has an accession number of CGMCC 22479.

9. The method according to claim 7, wherein a coding gene for the cellobiohydrolase protein Thph2 has a nucleotide sequence selected from the group consisting of the following (X1) to (X4):
(X1) the nucleotide sequence set forth in SEQ ID NO: 2;
(X2) a polynucleotide encoding the protein with the amino acid sequence set forth in SEQ ID NO: 1;
(X3) a DNA sequence having a homology of 90% or more with the nucleotide sequence set forth in SEQ ID NO: 2 and encoding a protein with a same function as a protein encoded by the nucleotide sequence set forth in SEQ ID NO: 2; and
(X4) a nucleotide sequence produced through hybridization with the nucleotide sequence set forth in SEQ ID NO: 2 under stringent conditions, wherein the stringent conditions are as follows: the hybridization is conducted in a 0.1×SSPE solution comprising 0.1% of SDS or a 0.1×SSC solution comprising 0.1% of SDS at 65° C., and membrane washing is conducted in a same solution as the hybridization is conducted.
